(19) United States
(12) Patent Application Publication
(10) Pub. No.: US 2020/0397959 A1
(43) Pub. Date: Dec. 24, 2020

(54) REMOTELY CONTROLLED SUCTION/IRRIGATION FOR ROBOTIC SURGERY

(71) Applicant: Vascular Technology, Incorporated, Nashua, NH (US)

(72) Inventors: Gary Douglas, Billerica, MA (US); Stephen Martone, Nashua, NH (US); David Regan, Pelham, NH (US); Nilendu Srivastava, Hollis, NH (US); Rachana Suchdev, Hollis, NH (US)

(73) Assignee: Vascular Technology, Incorporated, Nashua, NH (US)

(21) Appl. No.: 17/011,421

(22) Filed: Sep. 3, 2020

Related U.S. Application Data
(63) Continuation of application No. 15/610,911, filed on Jun. 1, 2017.
Application No. 15/610,911 is a continuation of application No. 13/469,654, filed on May 11, 2012, now abandoned.
(60) Provisional application No. 61/485,833, filed on May 13, 2011.

(51) Int. Cl. A61M 1/00

(52) U.S. Cl. CPC A61M 1/0084; A61B 2017/00212; A61M 1/0047; A61M 1/0058

(57) ABSTRACT

A system for the movement of fluids into and out of a surgical field is provided with a control module manipulated by a user; a valve controlled by the user via the control module, the valve controlling flow of a fluid; and a tube set having a proximal branch and a distal branch, wherein the proximal branch is opened and closed by the valve and at least a portion of the distal branch being flexible and a distal tip of the distal branch configured for manipulation within a surgical field. The valve is disposed at a distance to the surgical field and allows free access to tube by the user, Separate proximal branches for suction, irrigation, and insufflation are provided. One or more distal branches may be provided.

### CROSS REFERENCE TO RELATED APPLICATIONS

This application is a continuation of U.S. application Ser. No. 13/469,654, filed May 11, 2012 which claims the benefit of U.S. Provisional Application No. 61/485,833, filed May 13, 2011 Each of these applications is herein incorporated by reference in its entirety for all purposes.

### FIELD OF THE INVENTION

The invention relates to suction/irrigators for surgery and more particularly, to a remotely controlled suction irrigation device for use in surgical procedures.

### BACKGROUND OF THE INVENTION

In surgical procedures, it is often necessary to irrigate sterile solutions into and aspirate bodily or irrigant fluids out of the surgical field. Traditionally, suction/irrigation devices have been designed to function as hand held tools intended for use by the operating surgeon or an operative assistant. These hand held devices typically incorporate a valve mechanism which the surgeon manipulates manually to control suction and irrigation functions. A common valve configuration in use today is known as a “trumpet valve”. The trumpet valve consists of a button which can be manually depressed by the operator against a compression spring to engage a valve barrel; manual depression of the suction key allows for aspiration through the device, while depression of the irrigation button allows for the irrigation of fluids through the device.

Recently, robot assisted surgery has been increasingly employed by surgeons to perform technically challenging procedures in a minimally invasive fashion. In robot assisted surgery, the operating surgeon sits at a robotic console and remotely controls robotic arms within the surgical field to perform the surgery. An assistant surgeon is present at the patient's side to manipulate tools that cannot be controlled robotically. One such tool is the suction/irrigation probe.

Suction/irrigation devices that are currently employed in robot assisted surgery are devices that were designed for laparoscopic surgery. The assistant surgeon inserts a rigid laparoscopic suction/irrigation probe through an accessory port and then manually manipulates this probe within the surgical field. As mentioned above, the control buttons on this probe consist of a dual trumpet valve which must be manually pressed and depressed by the assistant surgeon, and cannot be manipulated by the lead surgeon seated at the robotic console. Thus, the lead surgeon must continuously instruct the assistant as to when, where and how to utilize the device throughout the surgical case.

The concept of a “band-held” suction/irrigation device can pose problems outside the realm of robot assisted surgery as well. In many open surgical procedures, the operating surgeon will often require an assistant to perform suction and/or irrigation within the surgical field while the surgeon is performing other surgical maneuvers (ex. suturing or dissection). In these instances, the surgeon will often have an instrument in each hand, preventing him/her from operating the valves on a traditional suction/irrigation device. In these instances, as well as in robot assisted surgery, it would be ideal for the surgeon to be able to place an appropriately designed suction/irrigation device within the surgical field and control the operation of this device in a “hands-free” or remote fashion.

An additional limitation posed by the use of trumpet valves or similarly actuated valve mechanisms is the propensity of these valves to become obstructed with particulate materials. The design of such valves not only limits the luminal size at the valve barrel, but also creates unnecessary surfaces/edges within the lumen that can cause turbulence and promote obstruction; these factors contribute to the creation of a “bottleneck” at the valve mechanism. An ideal control valve would operate externally to the flow path and allow for complete opening of the inner lumen, thus minimizing the potential for obstruction of flow.

Yet another limitation imposed by the valvular mechanisms that are currently used in suction/irrigation devices today is that they impede the ability to manipulate the flow path in instances of obstruction. Frequently, obstruction of a suction/irrigation device will occur at the valve assembly; however, most valve assemblies are either integrated into the flow stream or inaccessible to manipulation because of device design. A valve mechanism that would allow a user to removably manipulate the tubing at the valve/tubing interface to clear a nidus of obstruction or otherwise troubleshoot clotting at the valve interface would be an advance in the field.

Furthermore, the rigid design of the distal portion of a suction/irrigation probe inherently limits the operating range of this device. Laparoscopic devices are inserted into the operative field via a fixed port. The fixed port and the rigidity of the probe only allow for a limited range of motion for the probe within the surgical field. Thus, there are areas within the surgical field in which the rigid suction/irrigation probe cannot be employed. In these areas, alternative ports must be used to properly place the rigid probe and thus allow for suction/irrigation capability—this not only adds to the complexity of the case, but may also necessitate an additional surgical incision for the patient. This limitation is problematic not only in robot assisted surgery, but in other forms of minimally invasive/laparoscopic surgery as well.

In addition, currently employed suction/irrigation devices are typically single use devices and must be disposed of at significant cost. To reduce cost, an ideal disposable suction/irrigation device would limit the components that became contaminated with use, thus necessitating replacement of only a limited number of such components.

Moreover, suction and irrigation capabilities are only required intermittently during a surgical procedure. Thus, while in robot assisted procedures it may be possible to provide a robotic arm that gives the console surgeon the ability to apply suction and/or irrigation, this necessitates dedication of an arm to these functionalities. A suction/irrigation device that could be controlled by the console surgeon without necessitating dedicated use of a robotic arm would be an advance in the field.

What is needed, therefore, is a flexible suction/irrigation device which can be remotely controlled by a surgeon, including a surgeon who may be operating from a robotic console, which also ideally optimizes the valvular mechanism to minimize the incidence of obstruction and allow for clearance of the obstruction, and minimizes disposable components to minimize cost without compromising patient safety.

### SUMMARY OF THE INVENTION

One embodiment of the present invention provides a system for the movement of fluids into and out of a surgical field with a control module manipulated by a user, a valve controlled by the user via the control module, the valve controlling flow of a fluid; and a tube set having a proximal branch and a distal branch, wherein the proximal branch is opened and closed by the valve and at least a portion of the distal branch being flexible and a distal tip of the distal branch configured for manipulation within a surgical field. The valve is disposed at a distance to the surgical field and allows free access to tube by the user. Separate proximal branches for suction, irrigation, and insufflation are provided. One or more distal branch may be provided. Distal branches may be equipped with additional valves whereby they may be closed to allow flushing of the system during the surgical procedure to remove blockages in the suction line.

Another embodiment of the present invention provides such a system wherein the tip is a rigid tip.

A further embodiment of the present invention provides such a system wherein the tip has a length not longer than 2 inches.

Still another embodiment of the present invention provides such a system wherein the tip comprises a porous surgical mat.

A still further embodiment of the present invention provides such a system further comprising a remote controller by which the user manipulates the control module.

Yet another embodiment of the present invention provides such a system wherein the remote controller is selected from the group of remote controllers consisting of voice controllers, foot pedals, pneumatically activated controls, wirelessly transmitted controllers and touch sensitive hand controllers.

A yet further embodiment of the present invention provides such a system wherein the remote controller is integrated into a robotic surgical system control console.

Even another embodiment of the present invention provides such a system wherein valve in the valve is a solenoid valve.

An even further embodiment of the present invention provides such a system wherein the solenoid valve comprises a solenoid pinch valve.

Still yet another embodiment of the present invention provides such a system further comprising a pump or regulator whereby the flow of fluid is pressurized.

A still yet further embodiment of the present invention provides such a system wherein the tip of the distal branch is repositionable by a robotic arm.

Even yet another embodiment of the present invention provides such a system wherein the tip of the distal branch is repositionable by a surgical instrument.

An even yet further embodiment of the present invention provides such a system further comprising a second distal branch coupled to the proximal branch and terminating in a laparoscopic suction irrigation probe.

Even still another embodiment of the present invention provides such a system further comprising a valve whereby the distal branch may be occluded.

One embodiment of the present invention provides a flexible suction/irrigation probe, the probe comprising: a distal tip having a plurality of porosities for the passage of fluids and being configured to be manipulated by a surgical robot; and a flexible tube coupled to a proximal end of the distal tip.

Another embodiment of the present invention provides such a probe, the probe further comprising a vacuum source coupled to the tube.

A further embodiment of the present invention provides such a probe, the probe further comprising an irrigation source coupled to the tube.

Still another embodiment of the present invention provides such a probe further comprising a compressed air source coupled to the tube.

A still further embodiment of the present invention provides such a probe further comprising a valve whereby the lumen is occluded.

Yet another embodiment of the present invention provides such a probe further comprising a check valve disposed within the tube between the distal end and an irrigation source.

One embodiment of the present invention provides a fluid control valve for use in suction, insufflation and irrigation of a surgical field, the valve comprising: a remote control unit operated by a user; a fluid line accessible to the user during a surgical procedure and running from a fluid source to the surgical field, and, opened and closed by a valve controlling the passage of fluid through a lumen of the fluid line; the valve disposed remotely from a surgical field, and remotely controlled by the remote control unit.

Another embodiment of the present invention provides such a fluid control valve, wherein the valve is a pinch valve disposed around the fluid line such that when the pinch valve is activated, the pinch valve compresses the fluid line and occludes the lumen, the pinch valve being configured to avoid turbulence and clotting in fluid passing through the lumen.

A further embodiment of the present invention provides such a fluid control valve wherein the fluid source is a fluid source chosen from the fluid sources consisting of vacuum sources, compressed gas sources, carbon dioxide sources, irrigation liquid reservoirs, and saline supplies.

Still another embodiment of the present invention provides such a fluid control valve further comprising a second pinch valve controlling fluid flow through a second fluid line, the second pinch valve being configured to be open only when the pinch valve is closed.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIG. 1 is a block diagram illustrating a flexible suction irrigator configured in accordance with one embodiment of the present invention and manipulable by a robotic surgical grasper.

FIG. 2 is a block diagram illustrating a flexible suction irrigator system configured in accordance with one embodiment of the present invention.

FIG. 3 is a block diagram illustrating a fluid supply hookup for a flexible suction irrigator configured in accordance with one embodiment of the present invention.

FIG. 4 is a block diagram illustrating a valvular unit of a flexible suction irrigator system configured in accordance with one embodiment of the present invention

FIG. 5 is a block diagram illustrating a valvular unit of a flexible suction irrigator system configured in accordance with one embodiment of the present invention having pressure regulators and an insufflation line.

FIG. 6 is a block diagram illustrating a flexible suction irrigator system configured in accordance with one embodiment of the present invention.

FIG. 7 is a block diagram illustrating a detail of a flexible suction irrigator system valvular unit configured in accordance with one embodiment of the present invention.

FIGS. 8A-8G are block diagrams illustrating various tips for use with a flexible suction irrigator configured in accordance with one embodiment of the present invention.

FIG. 9 is an exploded perspective view of a suction irrigator tip configured in accordance with one embodiment of the present invention.

FIG. 10 is a perspective view of a suction irrigator surgical platform configured in accordance with one embodiment of the present invention.

FIG. 11 is a perspective view of a suction irrigator tubing set configured in accordance with one embodiment of the present invention.

FIG. 12 is a perspective view of a suction irrigator tubing set configured in accordance with one embodiment of the present invention with an assistant surgeon suction probe.

FIG. 13 is a perspective view of a suction irrigator hand control configured in accordance with one embodiment of the present invention.

### DETAILED DESCRIPTION

One embodiment of the present invention provides a suction/irrigation device for use during surgery that can be manipulated and controlled by a surgeon in a hands-free or remote fashion. The device consists of a flexible distal probe which can be brought into the operating field and a valvular unit which allows for the remote operation of suction and irrigation functions by the surgeon.

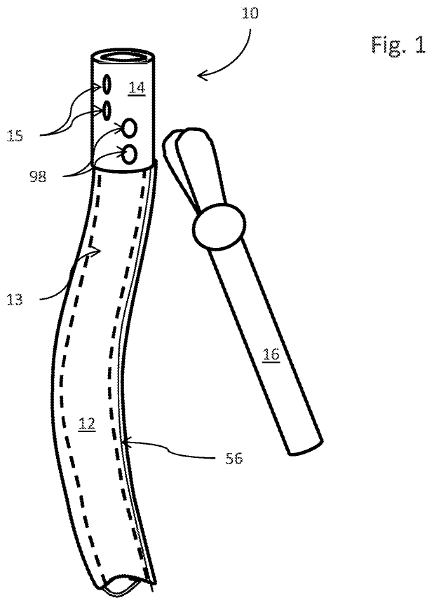

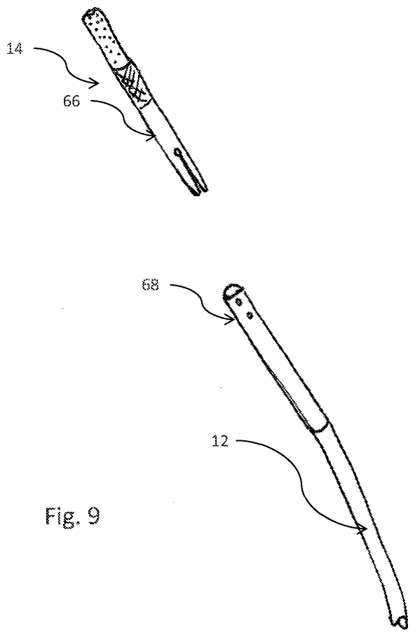

The flexible distal probe 10 illustrated in FIG. 1 consists of flexible tubing 12 with at least one lumen 13 that can be inserted into a surgical field and a graspable tip 14. The tubing 12 can be configured for insertion through a surgical port for use in minimally invasive or robot-assisted surgery. The graspable tip 14 in one embodiment is as simple as a hollow, rigid cylindrical attachment to the tubing with suction relief holes 15 which allows the surgeon to grasp the tip 14 with a surgical tool 16 and manipulate the flexible probe easily. The surgical tool 16 could comprise a tool integrated within a robotic arm. Tip 14 could be attached to tubing using an adhesive (in one embodiment the adhesive is biocompatible), a coupling sleeve, or any other mechanical means of attachment. In an alternative embodiment, as illustrated in FIG. 9, the tip 14 may be configured to be detachably affixed to the tubing with a compression joint, the joint being made up of complementary male 66 and female 68 fittings. Other detachable means of connecting tip 14 to tubing 12 could also be employed.

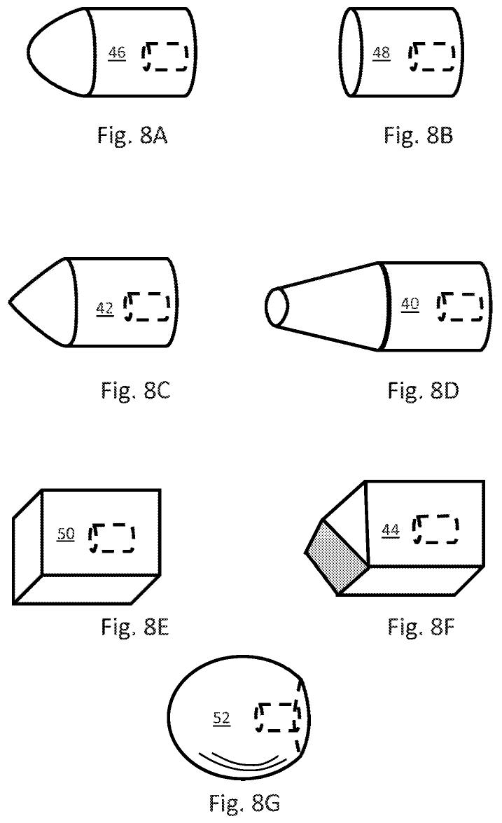

The graspable tip 14 can also take the form of a porous sintered or foam tip 14 which would provide for greater dissection capability of the invention. Possible tips may include a tip such as that described in U.S. application Ser. No. 12/797,067 filed Jun. 9, 2010 which is incorporated herein by reference for all purposes. Such tips, as illustrated in FIGS. 8A-8G can be conic 42, frustro-conic 40, wedge shaped 44, cylindrical 48, spherical 52, cubic 50 or some combination thereof 46. Alternatively, the graspable tip 14 could be fashioned by modifying the end of the flexible tubing 12 by processes such as shaping the tubing to allow for greater purchase on the tubing by the grasper 16 or by the introduction of suction relief holes through the wall of the tube, allowing for ease of use with a robotic arm without a separate tip.

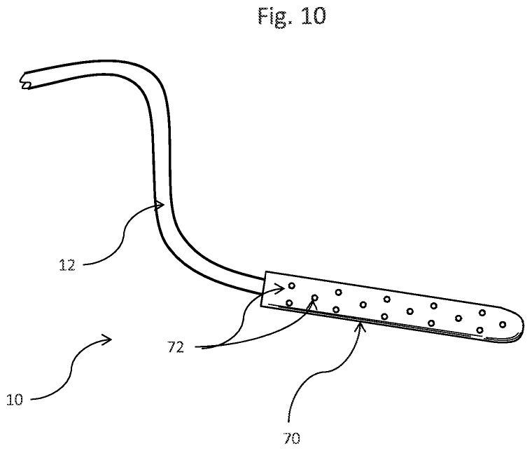

In another embodiment of the present invention, the tip 14 could comprise a porous surgical mat as illustrated in FIG. 10. In this embodiment, the flexible distal probe 10 consists of flexible tubing 12 and a porous, low profile, rigid, semi-rigid or flexible manifold 70 which can be constructed of any biocompatible material including but not limited to PVC, polytetrafluoroethylene (PTFE), polyurethane, polycarbonate, polyether ether ketone (PEEK), polyamides, nylon, silicone and polyether block amide (PEBAX). Processes which could be used to fabricate such a surgical mat include but are not limited to extruding, molding, forming, machining, selective laser sintering, stereolithography, and fused deposition modeling. This manifold/mat 70 is designed to be placed under or behind an anatomical structure(s) within the surgical field. The porosities 72 within the mat 70 allow for suction and irrigation to be delivered via the tubing 12, through the mat 70 and into the surgical field. The semi-rigid nature of the manifold 70 provides a stable surface upon which to perform surgical maneuvers including but not limited to anastomoses of microvasculature, nerve fascicles, and ligaments in reconstructive procedures, rejoining of ligated vas deferens tubules in vasectomy reversal procedures, and ligation of aberrant vasculature in varicocelectomy procedures.

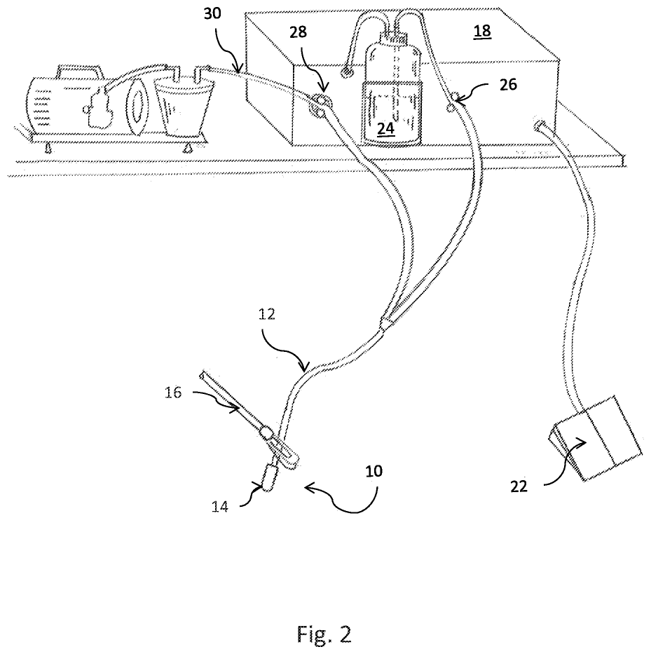

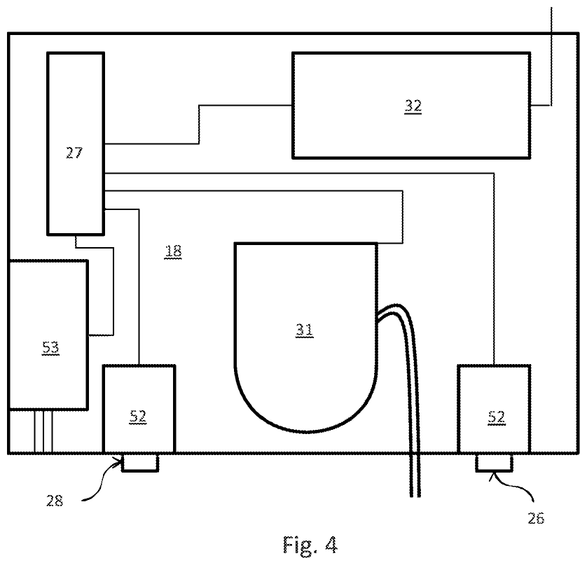

A valvular unit 18 as illustrated in FIG. 2 may be controlled through wired, wireless, or mechanical means. In one embodiment, the user may control the valvular unit 18 with a foot pedal 22. The user input via the valvular unit 18 allows the valvular unit 18 to actuate a supply of sterile saline or other irrigation fluid 24 and two valves 26, 28. A first valve 26 controls the flow of irrigation fluid from the supply source 24, while that of the second valve 28 controls a line 30 running from a vacuum source such as a vacuum pump or vacuum line. System 18, as seen in FIG. 4, may also be equipped with power supply 32 supplying power to the irrigation fluid supply pump 31 and actuators for the valves 26, 28. A bus bar or terminal block 27 receives power from the power supply 32 and distributes power to various components of the valvular unit 18.

The valvular unit 18 sits outside of the surgical field. The valvular unit 18 of one embodiment uses valves 26, 28 that are solenoid pinch valves 52 which are used to turn the suction and irrigation functions on and off. One skilled in the art will appreciate that other valves or switches may be employed, such as, mechanical solenoid valves. Power switches 53 may be provided to allow the user to turn on the valvular unit 18 and to switch between running and setup modes. The pinch valves can be controlled via various mechanisms including (but not limited) to a foot pedal 22, additional buttons at the robotic console finger controls, buttons 98 at and/or on the distal probe tip 12, as in FIG. 1, (which may require wiring 56 back to the control unit along the flexible tubing), voice activation or any other actuation signal communication forms.

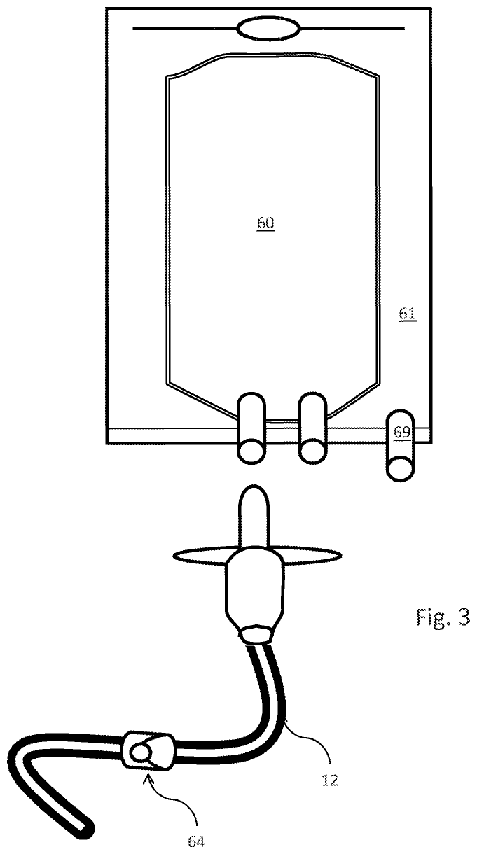

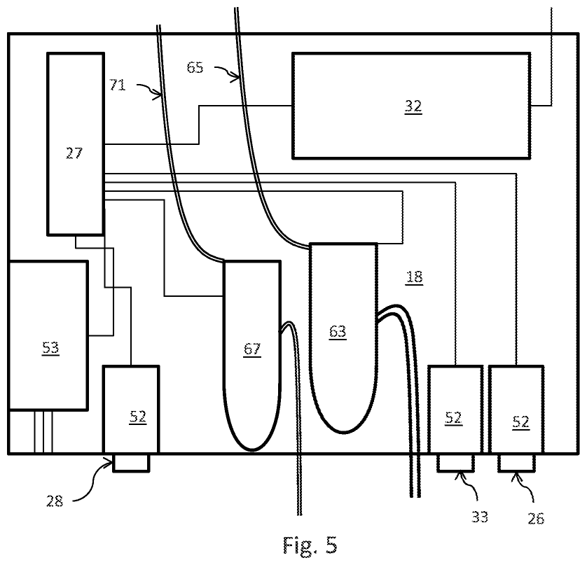

In addition, the valvular unit irrigation fluid supply 24 may include a pumping mechanism (ex. air pump) 31 to allow for “power” irrigation and/or gravity irrigation in the event of power failure. Alternatively, such a pump could be housed in a unit separate from the unit that allows for actuation of suction and/or irrigation capabilities. In one embodiment of the present invention, a detail of which is illustrated in FIG. 3, the system may be configured to provide irrigant from a fluid bag 60 (ie. such as that used for intravenous infusions) or other container separate from the valvular unit 18. Elevation of the fluid bag would allow for gravity fed irrigation. Alternatively, an inflatable pressure cuff 61 could be placed around the fluid bag and inflated via the air pump housed in the valvular unit 18 to allow for pressurized/powered irrigation. In another embodiment of the present invention, as shown in FIG. 5 the valvular unit 18 would comprise a connection 65 to an external pressurized air source such as the hospital central air supply. The valvular unit 18 would house a pressure regulator 63 with or without a safety pressure relief valve which would allow for the delivery of a constant air pressure to the inflatable pressure cuff via connector 69. As in FIG. 5, a similar system may be employed to provide insufflation capability wherein a gas, such as compressed air or carbon dioxide is the fluid supplied, from a gas supply 71 through a regulator 67 to a proximal branch and ultimately to the surgical field. In such a valvular unit, an additional valve 33 may be provided to control gas from the gas regulator 67. As used throughout the specification, fluid is used to indicate a substance in either a gas or liquid phase and any particulates or sold masses contained therein.

One embodiment of the present invention further provides at least one check valve 64 disposed within said flexible tube to prevent fluids from returning to the pump or from evacuated fluids being returned to the surgical field

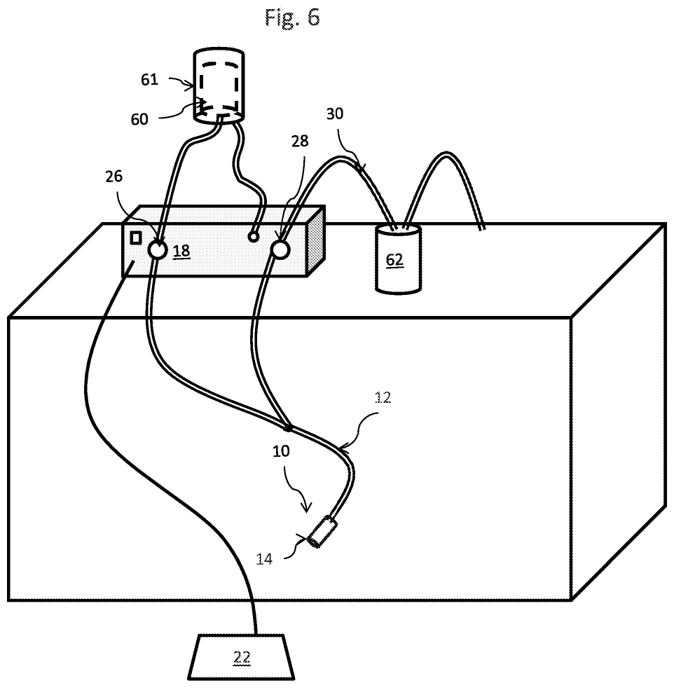

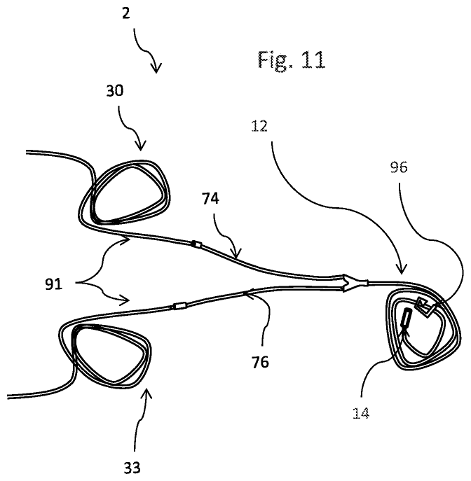

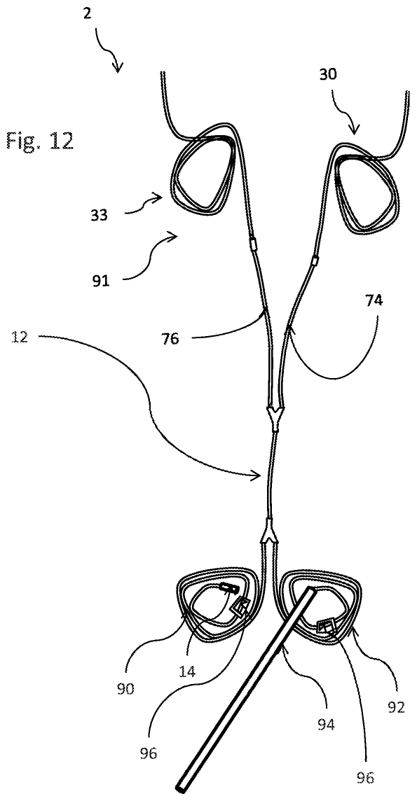

As illustrated in FIG. 6, the tubing set 2, illustrated in more detail in FIGS. 11 and 12, of the invention has a distal branch of tubing 12 (and possibly wiring, not shown) and a proximal branch of tubing that comes back to the valvular unit 18, passes through the solenoid pinch valves 26, 28 and then connects to both a suction source/container 62 and an irrigation source 60. In this manner, only the tubing set 2, and the container 62 for collection of the aspirant is exposed to contaminants. All other components of the valvular unit 18, including the solenoid pinch valves 26, 28, the irrigation pump 31 and/or pressure regulators 63, 67 and the power supply 32 can be reused. Such a design not only reduces costs but also minimizes the need to dispose of bulky disposable pump apparatus. Furthermore, the use of pinch valves is less prone to blockage than trumpet valves used in known systems, as pinch valves do not come into contact with the lumen and fluid flow path, thus minimizing turbulence within the flow stream.

As shown in FIGS. 11 and 12, the proximal branch 91 of tubing set 2 may comprise sections of compliant tubing (74, 76), such as silicone tubing, allowing for compression by the pinch valves. A tubing set 2 of one embodiment has at least one a distal branch configured for manipulation within the surgical field and a proximal branch outside of the surgical field and configured for connection with the valvular mechanism of the valvular unit, tubing sections 74 and 76 can be accessed and manipulated during a surgical procedure. For example, if tubing section 74 was to become clotted with blood clots aspirated during surgery, a user or assistant could place the unit in “setup” mode via control switch 53 (which would open both solenoid valves 26 and 28), lift pliant tubing section 74 out of the valvular mechanism, and mechanically dissolve the clot by applying manual pressure to the outer tubing surface to restore the suctioning functionality. Alternatively, a valve 93 or pinch clamp 96 could be closed and both solenoid valves 26 and 28 could be opened thereby forcefully flushing the clot from the selected area of the tubing set 2.

Tubing 12, according to one embodiment of the present invention may be tubing made from PVC, silicone or C-flex type tubing or other tubing with desired biocompatibility, flexibility and resistance to leakage or rupture even after a high number of flexure cycles. As illustrated in FIG. 11, conformable sections of tubing 74, 76 may be inserted within tubing of less compressible material to allow better action of valvular mechanisms incorporated into the system.

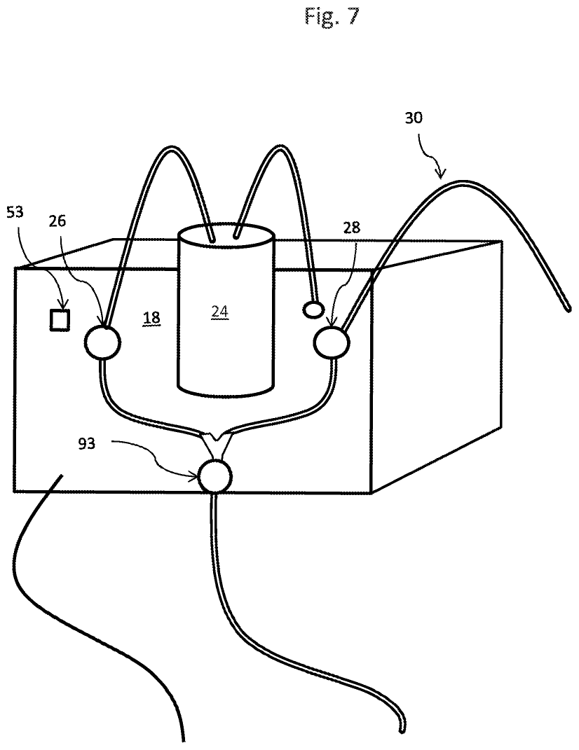

In one embodiment of the present invention, the placement of the valvular mechanism away from the surgical field also allows a user other than the surgeon to control suction, irrigation or blowing functions when necessary. In such an embodiment, valves 26 and 28 would comprise solenoid valves with push buttons which could be manually depressed to allow for opening and closing of the valves. This again would be especially important if a clot were to obstruct the flow of fluid within the system. Taking again the example above, if a clot was to develop in tubing section 74 thus prohibiting suctioning, nursing staff in the operating room could troubleshoot this problem via manual manipulation of the valvular unit. Besides employing the maneuver mentioned above, the nursing staff could also close off the pinch valve 96 or as in FIG. 7, an additional valve 93 on said valvular unit 18 that controls the distal branch, and then simultaneously press both the suction and irrigation control buttons on the valvular unit. This would force pressurized irrigant to flow preferentially from tubing limb 33 through tubing section 76 and up through tubing limb 30 via tubing section 74. This “purging” of the system could allow nursing staff to clear clots within the tubing set and particularly at the valvular interfaces if such clots were to develop. In an alternative embodiment, valve 93 could also be remotely controlled thus allowing a surgeon operating from a robotic console to purge the system remotely.

In one embodiment of the present invention, the console surgeon uses the robotic arms to grasp and manipulate the distal probe tip 14, and uses the foot pedal 22 (or other actuation controls such as voice activation, controls integrated into the robotic console, or buttons at the distal probe tip, pneumatically activated controls, and wirelessly transmitted controllers) to apply suction or irrigation via control of the solenoid valves. This design allows the console surgeon to independently control suction, irrigation and insufflation/blowing functions without the need for an assistant. In addition, the flexible design of the suction/irrigation probe allows the console surgeon to apply suction and/or irrigation to areas within the surgical field that would have been inaccessible with a rigid probe inserted via a fixed port. This flexible design also allows for complete freedom of motion at the probe tip, enabling the user to articulate the tip 180 degrees such that suction or irrigation could be aimed in a direction opposite to the orientation of the probe. Tubing materials such as braided or reinforced tubing could be used to allow for maximal “bending” of the probe tip without causing the formation of occlusive kinks within the tube lumen. This freedom of motion is especially useful in minimally invasive procedures when the suction/irrigation probe and robotic/laparoscopic camera are oriented/inserted from similar positions or angles within the surgical field. In these instances, complete freedom of motion at the probe tip would allow the surgeon to clean a soiled camera lens by aiming irrigation in a “backwards” direction.

In another embodiment of the present invention, a user in the room may, directly control the valvular unit, while allowing the console surgeon to manipulate the position of the tip within the surgical field. In an alternative embodiment of the present invention as seen in FIG. 12, the tubing set 2 is configured to provide two distal limbs 90, 92 such that suction and irrigation functionalities can be utilized by two surgeons using the same valvular unit. In one embodiment, limb 92 is equipped with a laparoscopic suction irrigator tip 94 for manual manipulation by an assistant surgeon, while limb 96 is equipped with a distal tip 14 for remote manipulation by a robotic surgeon using robotic graspers. Other tip types could be chosen for each of the distal limbs. Additional limbs 90 and 92 could be connected via a Y connector or other appropriate connector to form distal branch 12 of tubing set 2. In such an embodiment, pinch clamps 96 may be provided to prevent suction or irrigation to occur through an undesired tip. Pinch clamps 96 may be manual clamps as shown in FIG. 12, or may be powered as with valve 93 in FIG. 7.

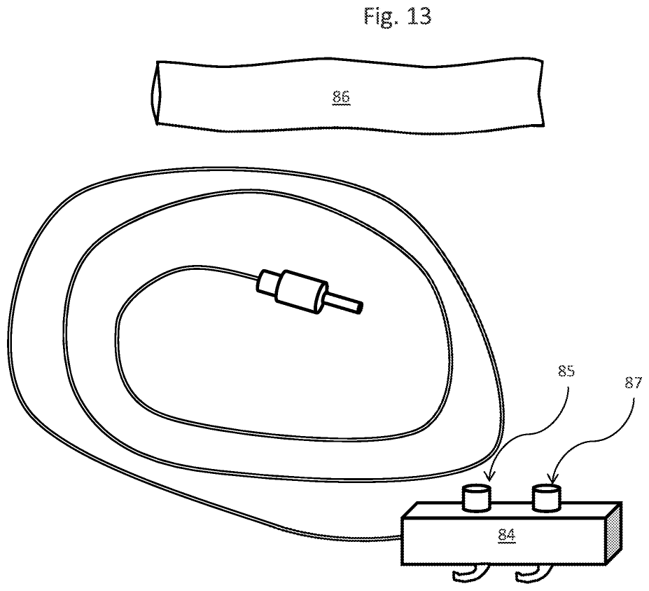

The laparoscopic suction irrigator tip 94, in one embodiment, could comprise a rigid tube with distal suction relief holes configured for insertion into a laparscopic port. In such an embodiment, a hand control 84 such as that illustrated in FIG. 13 may be used to allow the assistant surgeon to operate the valvular unit. Hand control unit 84 could comprise a small clip-on manifold with push buttons 85 and 87 which could connect to valvular unit 18 via wire 89. Depression of buttons 85 and 87 would allow the assistant surgeon to open and close valves 26 and 28 on valvular unit 18. The hand control 84 may be provided sterile or may be covered in a sterile disposable plastic sleeve 86 and may be configured for reuse or single use. Other means by which the assistant surgeon could operate the valvular unit 18 include but are not limited to a second foot pedal connection at the control unit, voice activation or any other actuation signal communication forms.

The foregoing description of the embodiments of the invention has been presented for the purposes of illustration and description. Each and every page of this submission, and all contents thereon, however characterized, identified, or numbered, is considered a substantive part of this application for all purposes, irrespective of form or placement within the application. This specification is not intended to be exhaustive or to limit the invention to the precise form disclosed. Many modifications and variations are possible in light of this disclosure.

1-17. (canceled)

18. An apparatus comprising:
a tube set configured for providing at least one of suction, irrigation, and insufflation to an anatomical region of interest, the tube set comprising at least one proximal branch and at least one distal branch;
a distal tip connected at a distal end of the at least one distal branch configured for insertion into a laparoscopic port;
a valvular unit connecting the at least one proximal branch to at least one valve, the at least one valve configured to control fluid flow through the tube set; and
a control module configured to receive input from a user,
wherein the distal end is configured to be grasped and manipulated using a grasper controlled by a robotic surgical system control console.

19. The apparatus of claim 18, wherein the distal end comprises a porous surgical mat.

20. An apparatus comprising:
a tube set configured for at least one of suction, irrigation, and insufflation, wherein the tube set comprises at least one proximal branch and at least one distal branch including a distal tip configured for insertion into an anatomical region of interest via a laparoscopic port;
a valvular unit connecting the at least one proximal branch to at least one valve, the at least one valve configured to control fluid flow through the tube set; and
a control module configured to receive input from a user.

21. The apparatus of claim 20, wherein the distal branch is configured with at least one of a graspable tip and a rigid tip.

22. The apparatus of claim 21, wherein the graspable tip is configured to allow for greater purchase on a distal end by a grasper.

23. The apparatus of claim 20, wherein the distal branch is configured to be switchable between a graspable tip and a rigid tip.

24. The apparatus of claim 20, wherein the at least one proximal branch comprises a first proximal branch configured for suction and a second proximal branch configured for at least one of irrigation and insufflation.

25. The apparatus of claim 24, further comprising a first pinch valve disposed around the first proximal branch and a second pinch valve disposed around the second proximal branch.

26. The apparatus of claim 20, wherein the at least one distal branch is configured to be grasped and manipulated using a remote controller integrated into a robotic surgical system control console.

27. The apparatus of claim 20, wherein the distal tip is configured to be detachably affixed to the at least one distal branch with a joint, the joint comprising a male fitting and a female fitting.

28. The apparatus of claim 20, wherein a portion of the tube set is removable and is configured to allow a user to manually clear an obstruction from the tube set.

29. The apparatus of claim 20, wherein a shape of the distal tip is selected from a list comprising conic, frustoconic, wedge-shaped, spherical, cylindrical, and cubic.

30. The apparatus of claim 20, wherein the distal tip comprises a porous surgical mat.

31. The apparatus of claim 20, wherein the at least one distal branch further comprises wiring.

32. The apparatus of claim 20, wherein the tube set is configured to provide pressurized irrigation.

33. The apparatus of claim 32, wherein pressurized irrigation is achieved through use of an inflatable pressure cuff surrounding a bag containing irrigant and held at constant pressure.

34. The apparatus of claim 32, wherein pressure is controlled via a control module by one or more of a pressure regulator and a valve.

35. The apparatus of claim 20, wherein the at least one distal branch comprises a check valve.

36. A method comprising:
providing a tube set configured for at least one of suction, irrigation, and insufflation, wherein the tube set comprises at least one proximal branch and at least one distal branch including a distal tip configured for insertion into an anatomical region of interest via a laparoscopic port;
grasping the distal tip with a grasper; and
manipulating the distal tip to a location for administration of the at least one of suction, irrigation, and insufflation.

37. The method of claim 36, further comprising controlling the grasper using a remote controller integrated into a robotic surgical system control console.

38. The method of claim 36, wherein the distal branch is configured to be switchable between a graspable tip and a rigid tip.

39. The method of claim 36, further comprising removing at least a portion of the tube set to allow a user to manually clear an obstruction from the tube set.
